SEQUENCE LISTING (3 sequences)

SEQ ID NO: 1 - protein, 803 aa, Clostridium Difficile
  Protein is designated Cwp84
MRKYKSKKLSKLLALLTVCFLIVSTIPVSAENHKTLDGVETAEYSESYLQYLEDVKNGDTAKYNGVIPFPHEMEGTTLRNKGRSSLPSAYKSSVAYNPMDLGLTTPAKNQGSLNTCWSFSGMSTLEAYLKLKGYGTYDLSEEHLRWWATGGKYGWNLDDMSGSSNVTAIGYLTAWAGPKLEKDIPYNLKSEAQGATKPSNMDTAPTQFNVTDVVRLNKDKETVKNAIMQYGSVTSGYAHYSTYFNKDETAYNCTNKRAPLNHAVAIVGWDDNYSKDNFASDVKPESNGAWLVKSSWGEFNSMKGFFWISYEDKTLLTDTDNYAMKSVSKPDSDKKMYQLEYAGLSKIMSNKVTAANVFDFSRDSEKLDSVMFETDSVGAKYEVYYAPVVNGVPQNNSMTKLASGTVSYSGYINVPTNSYSLPKGKGAIVVVIDNTANPNREKSTLAYETNIDAYYLYEAKANLGESYILQNNKFEDINTYSEFSPCNFVIKAITKTSSGQATSGESLTGADRYETAVKVSQKGWTSSQNAVLVNGDAIVDALTATPFTAAIDSPILLTGKDNLDSKTKAELQRLGTKKVYLIGGENSLSKNVQTQLSNMGISVERISGSDRYKTSISLAQKLNSIKSVSQVAVANGVNGLADAISVGAAAADNNMPIILTNEKSELQGADEFLNSSKITKSYIIGGTATLSSNLESKLSNPTRLAGSNRNETNAKIIDKFYPSSDLKYAFVVKDGSKSQGDLIDGLAVGALGAKTDSPVVLVGNKLDESQKNVLKSKKIETPIRVGGNGNESAFNELNTLLGK

SEQ ID NO: 2 - protein, 290 aa, Clostridium difficile
  Protein is designated FliC
MRVNTNVSALVANNQMGRNVNAQSKSMEKLSSGVRIKRAADDAAGLAISEKMRAQIKGLDQAGRNVQDGISVVQTAEGALEETGNILQRMRTLSLQSSNETNTAEERQKVADELLQLKDEVERISSSIEFNGKKLLDGSSTEIRLQVGANFGTNVAGTTNNNNEIKVALVNTSSIMSKAGITSSTIASLNVDGASGTDAAKQMVSSLDMALKELNTSRAKLGAQQNRLESTQNNLNNTIENVTAAESRIRDTDVASEMVNLSKMNILVQASQSMLAQANQQPQGVLQLLG

SEQ ID NO: 3 - protein, 506 aa, Clostridium difficile
  Protein is designated FliD
MSSISPVRVTGLSGNFDMEGIIEASMIRDKEKVNKAKQDQQIVKWKQEIYRDIIKESKNLYDKYLNGDSPNSITNKKAYSATRITSSDESIIVAKGSAGAEKINYQFAVSQMAEPAKVTIKLNSSDPIVQQFPPNASGASSLNIGGVNIPISEQDTTSTIVSKINSLCADNDIRASYSEMTGELIISRKQTGSSSDIDLKVIGNDSLAGQIASDNGITFTTDASGTKSAVVYGKNLEADVTDDQGRVTHISKEQNSFKIDNIDYNVNSKGSAKLVSVTDTEEATKNMKAFVDDYNALMDKVYGLVTTKKSKDYPPLTDEQKDDMTTEEIEKWEKKAKEGILRNDDELRAFVEDIQSMFFGDADTIIALRKLGISEHENYNKKGQISFNADTFSKALIDDSDKVYKALAGYSSNYDDKGMFEKLKKIVFEYSGSSASKLTKKAGMENSSSASQNVYSKQIAEQERNISRLVEKMNDKEKRLYAKYSALESLLNKYSSQMNYFSQAQG

(19) United States
(12) Patent Application Publication
(10) Pub. No.: US 2008/0222739 A1
(43) Pub. Date: Sep. 11, 2008

(54) Therapeutic Clostridium difficile antibody compositions

(76) Inventors: Lin Fang, Winnipeg (CA); Ronald R. Marquardt, Winnipeg (CA); R. Terence Sellen, Oak Bank (CA)

Correspondence Address: Robert WB BAILEY, BOX 2 STATION L, WINNIPEG, MB, R3H 0Z4, CA

(21) Appl. No.: 12/000,020

(22) Filed: Dec. 7, 2007

Related U.S. Application Data
(60) Provisional application No. 60/873,964, filed on Dec. 11, 2006.

(51) Int. Cl. C12P 21/00; C07K 16/00

(52) U.S. Cl. 800/4; 530/387.1

(57) ABSTRACT

The C. difficile proteins Cwp84, FliC and FliD, known to have conserved peptide sequences, were separately injected into female chickens, and the antibody rich egg yolks harvested. The egg yolks were then tested as the active ingredient in compositions against C. difficile bacteria.

Two copies of the sequence listing (Copy 1 Replacement and Copy 2 Replacement) on floppy disks, each containing the file named “sequence listing.txt” which is 13,757 bytes (measured in MS-DOS Windows 95 and 98) created on Apr. 23, 2008, together with a paper copy of the sequence listing, which are herein incorporated by reference.

### FIELD OF INVENTION

This invention is directed to antibody compositions against bacteria, specifically including Clostridium difficile. C. difficile is believed to be a primary infective agent in iatrogenic diarrhea, and sudden infant death syndrome in humans and is thought to be widespread in animal husbandry. It is believed to be a common cause of infection in hospitals. These antibody compositions have been tested in vitro against C. difficile.

### BACKGROUND

C. difficile has a number of characteristic proteins, notably SlpA, the so-called surface proteins, often referred to as P36 and P47, which are produced by a single gene as a single protein and then split, FliC (the main flagellum component) and FliD (the flagellum tip or cap protein), Cwp84 (a cell wall protein believed to be a protease and perhaps essential to the bacterial metabolism), Cwp66 (another cell wall protein).

For an antibody composition to be effective against a bacteria, it must interact with a protein, usually an outermembrane protein or a toxin, specific to that bacteria. Although this begs the question since bacteria are to some extent defined by their specific proteins, themselves expressed by bacterial DNA sequences, the composition, containing one or more antibodies must interact with a particular protein of the organism. Although proteins have a huge number of sequential aminoacid possibilities, as indicated below, most proteins having a particular function in a particular bacteria will have one or more common sequences of aminoacids. These sequences are known as conserved sequences, and should be present in every protein of this type in the species of bacteria. When a protein is used as an antigen to generate antibodies it is not necessary to use the entire protein; it is possible to use a fragment of the protein capable of generating an immune response, the fragment must form a conserved sequence of the sequence to be effective.

### PRIOR ART

As noted above C. difficile has six surface proteins, which were considered. In US Patent Application Publication 2003/0054009, to Windle et al., 20 Mar. 2003, teaches two SlpA sequences (No. 1 and 2), one for each SlpA protein, suitable to generate vaccines. Further taught are six sequences (No. 3, 4, 5, 6, 7 and 8) for the entire SlpA before cleavage for six strains of C. difficile. It does not appear from Windle that vaccines derived from these sequences (No. 1 and 2) corresponding to P47 and P36, are effective. Probably they were not, because the aminoacid protein sequences used were not common to every P36 and P47 protein, that is the sequence was heterogenous, to be effective the sequence must be homogenous, that is present in conserved C. difficile proteins.

More recently Pechine et al. tested 17 isolates from infected human patients, the proteins tested were FliC, FliD, Cwp66, both C and N terminal domains, and Cwp84, N terminal. Details of the generation of protein sequences, but not the actual sequences are given. FliC and FliD were detected (by antibody reaction) in 15 of 17 isolates, N-terminal Cwp66 was detected in all isolates, C-terminal Cwp66 was detected in 12 of 17 isolates, Cwp84 was detected in all isolates. It was confirmed that there is relatively little genetic variability in FliC and FliD, Cwp66 showed considerable genetic variation, especially at the C-terminal, while Cwp84 appeared uniform. Pechine et al. state that SlpA proteins are useful for phenotyping C. difficile, that is distinguishing genetic strains. The cwp66 gene (as opposed to Cwp66 protein) both 5′ and 3′ parts show high variability, while the 3′ part is known to be highly variable also, and thus the Cwp66 protein is suitable material for genetic analysis, and not for use as an antigen.

Pechine et al., conclude that Cwp84, FliC, and FliD proteins, or aminoacid (peptide) sequences thereof may be suitable antigens against C. difficile, which can be incorporated into a vaccine.

In practice C. difficile is difficult to vaccinate against since it operates against (or in) the gut lining of mammals and humans, and this zone is remote from the bloodstream of the animal. That is antibodies in the animal's bloodstream do not interact with C. difficile directly and thus do not easily affect the infection, which continues unabated.

As the above three proteins are surface proteins, and homogenous, they are exposed wherever C. difficile operates. Thus antibodies thereto can be expected to interact with it on contact. As this is acknowledged to be easier in the intestine, use of these antibodies in therapeutic compositions, orally dispensed in food or drink ingested by the animal or human, seemed feasible. Antibodies for internal proteins are less likely to be effective.

It was therefore decided to generate antibodies in avian egg yolk, by injecting the proteins Cwp84, FliC and FliD into bird species, typically hens. As will be understood by those skilled in the art, selected sequences from these proteins could possibly also be used as antigens to generate effective antibodies.

### DESCRIPTION OF THE INVENTION

Full protein sequences are given in the appendices, for Cwp84 (Sequence No. 1) FliC (Sequence No. 2) and FliD (Sequence No. 3). As will be understood by those skilled in the art, these sequences are the entire aminoacid sequence for samples of these C. difficile proteins, that is other similar but not identical sequences may also be defined as Cwp84, FliC, FliD proteins, however the differences are generally thought small, insignificant and negligible in their effects.

These three proteins are generated by known developed scientific techniques until a sufficient amount of each protein is available, which is then injected singly by known techniques into female chickens (hens). The hens develop specific antibodies to the proteins, which inter alia are found in the yolks of eggs laid by the hens. The eggs and their yolks are then harvested. The yolks are then treated to provide an antibody rich composition, suitable for use in the passive control of C. difficile. These compositions were then tested against C. difficile as indicated in the experimental data and found to have statistically significant effects. The next stage would be to add these compositions in suitable batches to food, liquid or solid, which then is then orally ingested by a patient, human or animal (usually, but not exclusively, in the sense of terrestrial mammal), and assess the clinical results, to determine the in vivo effects. One or more antibody compositions may be combined under these circumstances as an effective treatment.

As known to those skilled in the art, the polypeptide preferably consists of the entire sequence of Cwp84, FliC or FliD, or at least comprises the entire sequence of Cwp84, FliC, or FliD, thus guaranteeing the presence of every aminoacid in the sequence. Often satisfactory antibodies can be generated using a protein having an aminoacid sequence substantially identical to that of a particular protein. Sometimes effective antibodies can be generated by proteins having at least about 60% or 70% homology with a particular protein, that is at least 60 or 70% of the aminoacids are identical and in the same relative positions or order. Further sometimes a homologue, analogue or derivative of these proteins will generate effective vaccines. It must be understood that effective proteins must include conserved sequences which themselves are immunogenic fragments, and it may be difficult to identify such effective fragments without experiment.

C. difficile's general method of infection is by oral ingestion and attacking through and infesting the gut wall. Injection of a vaccine, into vein or muscle, will not usually cause antibodies generated by the vaccine to reach the site of infection which is the inflamed, damaged or destroyed gut wall remote from blood vessels in time or quantity to halt the infection. Feeding patients the antibodies in food will both effectively prevent C. difficile from colonising the gut wall, and neutralize C. difficile in the process of colonisation, by binding the proteins Cwp84, FliC, FliD and thus rendering them ineffective. It is believed that the six surface proteins noted above function to attach to the gut wall, and that C. difficile being unable to do so in the presence of antibodies will be unable to infect patients.

It is thought that the above noted proteins Cwp84, FliC, FliD, are present in other Clostridium species, and possibly other bacterial genera. If so the antibodies developed will be of broader effect and significance than C. difficile, alone.

In one broad aspect the invention is directed to a method of generating antibodies in avian egg yolk by injecting a vaccine comprising proteins selected from the group consisting of Cwp84, FliC and FliD of Clostridium difficile into female members of bird species and collecting the egg yolks. Preferably the bird species is chickens. The protein may be Cwp84, more preferably Cwp84 has Sequence No. 1. The protein may be is FliC, more preferably FliC has Sequence No. 2. The protein may be FliD, more preferably FliD has Sequence No. 3. Polypeptide fragments of these proteins may function as antigens.

In a second broad aspect the invention is directed to a antibody composition against C. difficile comprising as active ingredient at least one egg yolk component generated in avian egg yolk by injecting proteins selected from the group consisting of Cwp84, FliC and FliD of Clostridium difficile into female members of bird species and collecting the egg yolks. Preferably the bird species is chickens. The protein may be Cwp84, more preferably Cwp84 has Sequence No. 1. The protein may be is FliC, more preferably FliC has Sequence No. 2. The protein may be FliD, more preferably FliD has Sequence No. 3. Polypeptide fragments of these proteins may function as antigens.

### DESCRIPTION OF THE PREFERRED EMBODIMENTS

The proteins Cwp84, FliC, FliD prepared by standard techniques were injected using known techniques into hens (female chickens) to create antibodies of the IgY type. The eggs of the chickens were then harvested. The egg yolks were then separated and dried, and tested for the presence of antibodies. The dried egg yolk antibody composition was then tested as indicated below against C. difficile.

C. difficile (ATCC 43255) Adherence Inhibition Assay

The procedure is as follows:

DAY 1: Trypsinize a confluent T84 T-25 flask by first washing with 5 mL FC wash (+EDTA). Add 2 mL of 0.25% Trypsin +EDTA, wash and decant off. Next, add 2 mL of 0.25% Trypsin +EDTA, place in 37° C. incubator for 2 mins, then pipette off 1.5 mL. Place flask in incubator once again, until all lift from the bottom of the flask (DO NOT hit flask, to avoid clumping). Add 6 mL of DMEM/F12 +5% FBS (w/v) (the cell culture “media” ) and seed cells to sub-confluency (⅛ dilution) in a 24-well (all samples are tested in ntriplicate).

DAY 2: Reduce 1×10 mL flask of BHI in anaerobic chamber overnight.

DAY 3: Inoculate 20 uL of C. difficile ATCC 43255 spores into the reduced BHI falsk. Incubate in anaerobic chamber for 24 hours (OD600 should be ˜0.15 and ˜1×10⁹ CFU/mL according to previous growth curves, adjust if necessary if bacteria are still in log phase).

DAY 4: Harvest bacterial cells by spinning at 6000×g for 10 minutes and resuspend in 4 mL media.

In a 96-well plate add 100 uL of 20 mg/mL FliC, FliD, Cwp84, and pooled (mixed FliC, FliD, and Cwp84), partially purified IgY or control IgY (each inhibitor tested in triplicate). Next add 100 uL of C. difficile culture. Mix well, pipetting up and own carefully, so as not to shear the cells and incubate in anaerobic chamber for 1 hour.

Add 800 uL of media to each well of the T84 24-well plate. Next add 200 uL of the inhibitor/bacterial cells mixture (a 1/10 dilution of bacteria i.e.: ˜1×10⁸ bacterial cells/mL).

Incubate for 3 hours at 37° C. in anaerobic chamber. Wash cells three times in nsteril 1×PBS.

Trypsinize cells with 0.25% trypsin containing 0.53 mM EDTA, and plate in triplicate on Y plates.

Incubate for 72 hours.

Count colonies from triplicate dilutions and plot as “percent” total inoculum bound to T84 cells.

Results

| | Percent of | | | |
|---|---|---|---|---|
| | Inoculum | | | p value |
| | Bound | | | (compared |
| | to Cells | Standard | Sample | to IgY |
| | (Mean) | Deviation | Size | control) |
| Normal (1 mg/mL) | 0.156 | 0.031 | 9 | 0.098 |
| IgY Control (1 mg/mL) | 0.119 | 0.025 | 9 | — |
| FliC IgY (1 mg/mL) | 0.0799 | 0.025 | 9 | 0.059 |
| FliD IgY (1 mg/mL) | 0.0309 | 0.017 | 9 | 0.005 |
| Cwp84 IgY (1 mg/mL) | 0.0661 | 0.0062 | 9 | 0.016 |
| Pooled IgY (1 mg/mL) | 0.0479 | 0.012 | 7 | nyexit 0.013 |
| FliC FliD Cwp84 | | | | |

As can be seen the antibodies inhibit the observed “percent” of bound cells substantially. Statistically FliD IgY is significant at the 1% level, while Cwp84 and pooled IgY is sigificant at the 5% level, FliC IgY is significant at more than 5% and less than 10% and therefore at the borderline of probable significance.

As those skilled in the art would realize these preferred described details and materials and components can be subjected to substantial variation, modification, change, alteration, and substitution without affecting or modifying the function of the described embodiments.

Although embodiments of the invention have been described above, it is not limited thereto, and it will be apparent to persons skilled in the art that numerous modifications and variations form part of the present invention insofar as they do not depart from the spirit, nature and scope of the claimed and described invention.

1. Method of generating antibodies in avian egg yolk by injecting a vaccine comprising proteins selected from the group consisting of Cwp84, FliC and FliD of Clostridium difficile into female members of bird species and collecting the egg yolks.

2. Method of claim 1 wherein said bird species is chickens.

3. Method of claim 1 wherein said protein is Cwp84.

4. Method of claim 3 wherein said protein has Sequence No. 1.

5. Method of claim 1 wherein said protein is FliC.

6. Method of claim 5 wherein said protein has Sequence No. 2.

7. Method of claim 1 wherein said protein is FliD.

8. Method of claim 7 wherein said protein is Sequence No. 3

9. Antibody composition against C. difficile comprising at least one egg yolk component produced by the method of claim 1.

10. Antibody composition against C. difficile comprising at least one egg yolk component produced by the method of claim 2.

11. Antibody composition against C. difficile comprising at least one egg yolk component produced by the method of claim 3.

12. Antibody composition against C. difficile comprising at least one egg yolk component produced by the method of claim 4.

13. Antibody composition against C. difficile comprising at least one egg yolk component produced by the method of claim 5.

14. Antibody composition against C. difficile comprising at least one egg yolk component produced by the method of claim 6.

15. Antibody composition against C. difficile comprising at least one egg yolk component produced by the method of claim 7.

16. Antibody composition against C. difficile comprising at least one egg yolk component produced by the method of claim 8.

17. Antibody composition of claim 9 comprising antibodies generated by Cwp84 having Sequence No. 1.

18. Antibody composition of claim 9 comprising antibodies generated by FliC having Sequence No. 2.

19. Antibody composition of claim 9 comprising antibodies generated by FliD having Sequence No. 3.
